(19) United States
(12) Patent Application Publication
(10) Pub. No.: US 2018/0049631 A1
(43) Pub. Date: Feb. 22, 2018

(54) LIGHT SOURCE APPARATUS

(71) Applicant: HOYA CORPORATION, Tokyo (JP)

(72) Inventors: Hidenori TAKUSHIMA, Saitama (JP); Kunihiko ONOBORI, Tokyo (JP); Fumika YOKOUCHI, Tokyo (JP)

(73) Assignee: HOYA CORPORATION, Tokyo (JP)

(21) Appl. No.: 15/552,607

(22) PCT Filed: Oct. 21, 2016
(86) PCT No.: PCT/JP2016/081230

§ 371 Date: Aug. 22, 2017

(30) Foreign Application Priority Data
Nov. 17, 2015 (JP) 2015-224578

(51) Int. Cl. A61B 1/06; A61B 1/00; F21V 14/04; F21V 9/00; F21V 13/08; H04N 5/225

(52) U.S. Cl. CPC A61B 1/0638; A61B 1/0684; A61B 1/00004; A61B 1/00045; F21Y 2115/10; F21V 9/00; F21V 13/08; H04N 5/2256; F21V 14/04

(57) ABSTRACT

A light source apparatus that is provided includes: a light source; a switch for switching the light path of irradiation light emitted by the light source between a first light path and a second light path; and an optical filter that is fixedly arranged in the first light path and filters irradiation light propagating along the first light path into light in a specific wavelength region.

### TECHNICAL FIELD

The present invention relates to a light source apparatus for irradiating a subject with light.

### BACKGROUND ART

Endoscope systems that can capture special images are known. A specific configuration of this type of endoscope system is disclosed in WO 2012/108420 pamphlet (called “Patent Document 1” hereinafter), for example.

The endoscope system disclosed in Patent Document 1 includes a light source apparatus that is provided with a rotating filter. The rotating filter is an optical filter that allows only light in a specific wavelength region to pass, and rather than having a simple disk shape, has a special shape in which a portion of the outer circumferential region is cut away. A controller drives the rotating filter to rotate at a constant rotation period such that the optical filter portion and the cutaway portion successively enter the light path of irradiation light, and an image of biological tissue formed by irradiation light that passed through the optical filter portion and an image of biological tissue formed by irradiation light that passed through the cutaway portion (i.e., unfiltered irradiation light) are successively captured. The controller generates one observation image based on captured image data regarding the biological tissue irradiated by irradiation light that passed through the optical filter portion, generates another observation image based on captured image data regarding biological tissue illuminated with unfiltered irradiation light, and displays these two types of generated observation images side-by-side on the display screen of a monitor.

### SUMMARY OF INVENTION

Silk lines for detecting the rotation position of the rotating filter are printed on the central portion of the rotating filter disclosed in Patent Document 1. However, the silk lines are extremely small, and therefore there is a problem in that the rotation position of the rotating filter cannot be precisely detected if there is even a slight error in the silk lines.

The present invention was achieved in light of the above-described circumstances, and an object of the present invention is to provide a light source apparatus that is suited to irradiating a subject with two types of irradiation light that have different wavelength regions.

A light source apparatus according to an embodiment of the present invention includes: a light source; a switching means for switching a light path of irradiation light emitted by the light source between a first light path and a second light path; and an optical filter that. is fixedly arranged in the first light path and filters irradiation light propagating along the first light path into light in a specific wavelength region.

Also, in the embodiment, of the present invention, a configuration is possible in which the switching means alternatingly switches the light path of irradiation light between the first light path and the second light path in accordance with a timing synchronized with a predetermined imaging cycle.

Also, in the embodiment of the present invention, a configuration is possible in which the switching means has a light path changing means capable of being inserted into the light path of irradiation light. In this configuration, the irradiation light enters the second light path when the light path changing means is inserted into the light path of irradiation light, and the irradiation light enters the first light path when the light path changing means is removed from the light path of irradiation light.

Also, in the embodiment of the present invention, the light path changing means is a reflecting member that bends the light path of irradiation light, for example.

Also, in the embodiment of the present invention, a configuration is possible in which the switching means inserts the light path changing means into the light path of irradiation light or removes the light path changing means from the light path of irradiation light by shifting the light path changing means in a direction orthogonal to the light path of irradiation light.

Also, in the embodiment of the present invention, a configuration is possible in which the switching means inserts the light path changing means into the light path of irradiation light or removes the light path changing means from the light path of irradiation light by rotating the light path changing means about a predetermined shaft on which the light path changing means is supported.

Also, a light source apparatus according to an embodiment of the present invention may include a plurality of light sources. A first light source that emits first irradiation light and a second light source that emits second irradiation light, for example, are included among the plurality of light sources. In this case, when the light path of irradiation light is switched between the first light path and the second light path by the switching means, the second light source is accordingly switched between on and off states.

According to the embodiment of the present invention, a light source apparatus that is suited to irradiating a subject with two types of irradiation light that have different wavelength regions is provided.

### BRIEF DESCRIPTION OF DRAWINGS

FIG. 1 is a block diagram showing a configuration of an electronic endoscope system according to an embodiment of the present invention.

FIG. 2 is a diagram showing a spectral intensity distribution of LEDs included in the electronic endoscope system of the embodiment of the present invention.

FIG. 3 is a perspective view of a movable unit included in the electronic endoscope system of the embodiment of the present invention.

FIG. 4 is a diagram showing spectral characteristics of a narrow-band light filter included in the electronic endoscope system of the embodiment of the present invention.

FIG. 5 is a diagram for assisting a description of operations of the electronic endoscope system in various observation modes.

FIG. 6 is a diagram schematically showing a configuration of a movable unit according to a variation of the embodiment of the present invention.

FIG. 7 is a perspective diagram showing a configuration of a mirror and an actuator included in the movable unit according to a variation of the embodiment of the present invention.

### DESCRIPTION OF EMBODIMENTS

Hereinafter, an embodiment of the present invention will be described with reference to the drawings. Note that an electronic endoscope system is taken as an example of an embodiment of the present invention in the following description.

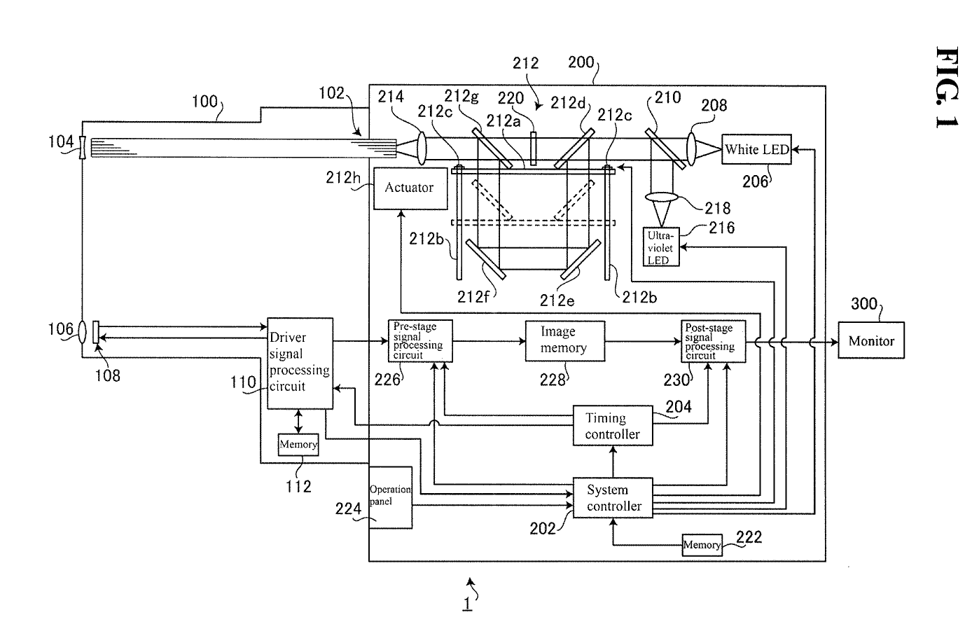

FIG. 1 is a block diagram showing the configuration of an electronic endoscope system 1 according to an embodiment of the present invention. As shown in FIG. 1, the electronic endoscope system 1 is a system specialized for medical use, and includes an electronic endoscope 100, a processor 200, and a monitor 300.

The processor 200 includes a system controller 202 and a timing controller 204. The system controller 202 executes various programs stored in a memory 222 and performs overall control of the electronic endoscope system 1. Also. the system controller 202 is connected to an operation panel 221. The system controller 202 changes operations of the electronic encloscope system 1 and parameters for various operation in accordance with instructions from an operator that are input using the operation panel 224. One example of an instruction input by an operator is an instruction for switching the observation mode of the electronic endoscope system 1. Examples of observation modes include a normal observation mode, a special observation mode, and a twin observation mode. The timing controller 204 outputs a clock pulse, which is for adjustment of the timing of the operations of portions, to circuits in the electronic endoscope system 1.

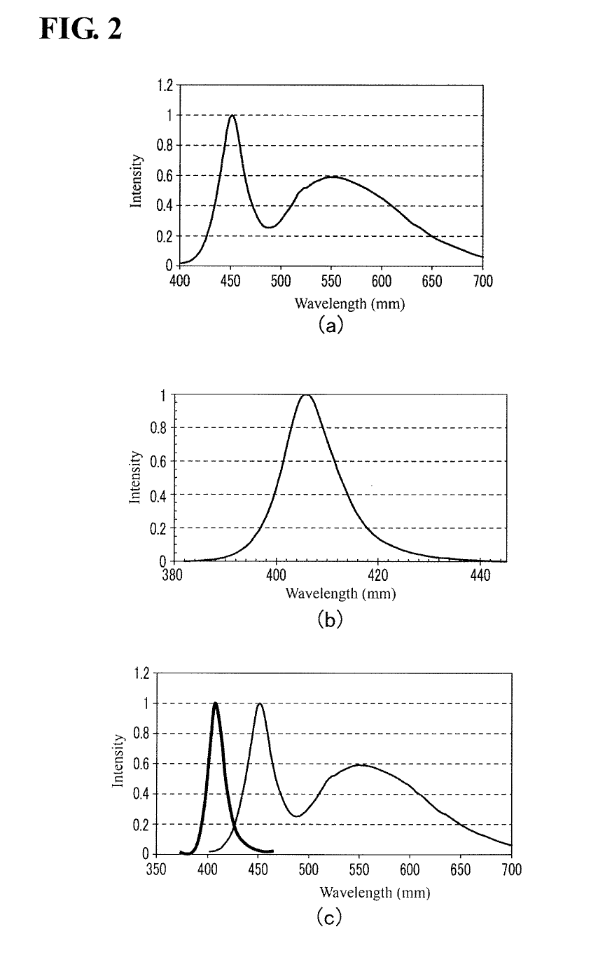

The processor 200 includes multiple LEDs (Light Emitting Diodes) as examples of light sources. Specifically, the processor 200 includes a white LED 206. FIG. 2(a) shows an example of the spectral intensity distribution of the white LED 206. As shown in FIG. 2(a), the white LED 206 is a so-called pseudo white light source that has an uneven emission spectrum. White light emitted by the white LED 206 passes through a collimator lens 208 and a dichroic mirror 210 in this order, and then enters a movable unit 212.

The processor 200 also includes an ultraviolet LED 216. FIG. 2(b) shows an example of the spectral intensity distribution of the ultraviolet LED 216. As shown in FIG. 2(b), the ultraviolet LED 216 is a light source that. emits only light in the ultraviolet region. Ultraviolet light emitted by the ultraviolet LED 216 passes through a collimator lens 218, is reflected by the dichroic mirror 210, and enters the movable unit 212.

The movable unit 212 operates as a switching means for switching the light path of light emitted by the light sources, and as shown in FIG. 1, includes a movable mount 212a, a linear shaft 212b, linear bushes 212c, a first mirror 212d, a second mirror 212e, a third mirror 212f, a fourth mirror 212g, and an actuator 212h. The mirrors inside the movable unit 212 function as light path changing means that can enter and exit the light path of light emitted by the light sources.

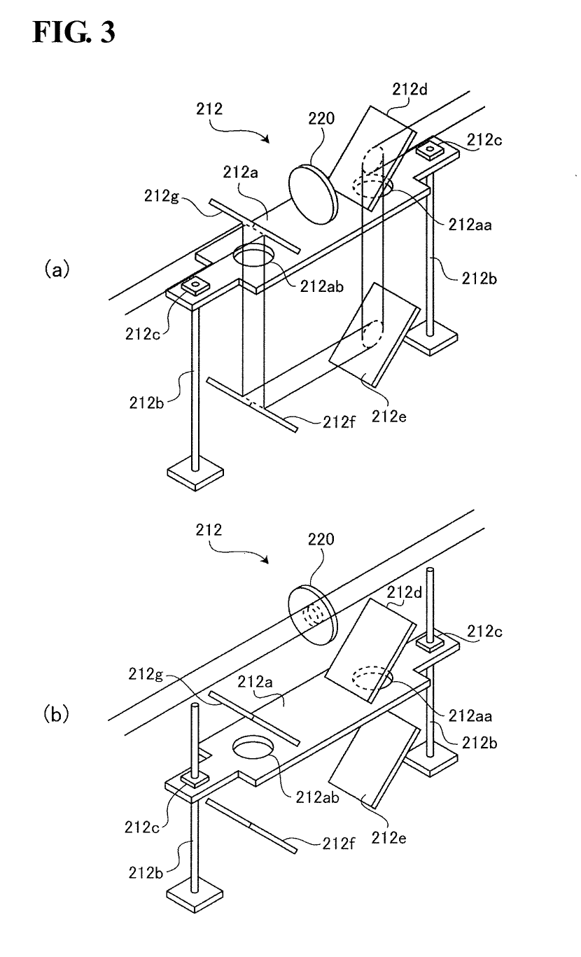

FIG. 3 shows a perspective view of the movable unit 212. Note that for the sake of convenience in FIG. 3, support members that support the various constituent elements of the movable unit 212 have been omitted from the illustration as appropriate, and the actuator 212h has also been omitted from the illustration.

As shown in FIG. 3, the linear bushes 212c are attached to the upper surface of the movable mount 212a. The linear shafts 212b, which are fixed to the case of the processor 200, guide the linear bushes 212c in a straight line, and thus the movable mount 212a shifts in the vertical direction (the lengthwise direction of the linear shafts 212b) inside the case. Note that the lengthwise direction of the linear shafts 212b is orthogonal to the light path of white light that passed through the dichroic mirror 210 (or ultraviolet light reflected by the dichroic mirror 212).

The first mirror 212d and the fourth mirror 212g are attached to the movable mount 212a, and shift in the vertical direction integrally with the movable mount 212a inside the case of the processor 200. In contrast., the second mirror 212e and the third mirror 212f are attached to the case, and have fixed positions in the case. Also, a narrow-band light filter 220, which is an example of an optical filter, is also attached to the case, and has a fixed position in the case. The narrow-band light filter 220 is shaped as a simple disk, for example.

When the movable mount 212a is shifted upward by the actuator 212h, the first mirror 212d is inserted into the light path of white light (or ultraviolet light) (see the first mirror 212d indicated by solid lines in FIG. 1, and see FIG. 3(a)). Hereinafter, for the sake of convenience in the description, the state in which the first mirror 212d has been inserted into the light path will be referred to as the “entered light path state”.

In the entered light path state, in order to circumvent the narrow-band light filter 220 located between the first mirror 212d and the fourth mirror 212g, white light (or ultraviolet light) that is incident on the first mirror 212d is reflected by the first mirror 212d, passes through a hole 212aa formed in the movable mount 212a, is reflected by the second mirror 212e and the third mirror 212f in this order, passes through a hole 212ab formed in the movable mount 212a, is reflected by the fourth mirror 212g, and then enters the condensing lens 214 arranged in the stage after the movable unit 212.

On the other hand, when the movable mount 212a is shifted downward by the actuator 212h, the first mirror 212d and the fourth mirror 212g are removed from the light path of white light (or ultraviolet light) (see the first mirror 212d indicated by dashed lines in FIG. 1, and see FIG. 3(b)). Hereinafter, for the sake of convenience in the description, the state in which the first mirror 212d has been removed from the light path will be referred to as the “exited light path state”.

In the exited light path state, white light emitted by the white LED 206 (or ultraviolet light emitted by the ultraviolet LED 216) passes through the narrow-band light filter 220 and enters the condensing lens 214.

In this way, in the entered light path state, unfiltered light (light that substantially has the same spectral intensity distribution as when emitted from the LED) enters the condensing lens 214, whereas in the exited light path state, light filtered by the narrow-band light filter 220 enters the condensing lens 214. Hereinafter, for the sake of convenience in the description, the light path that circumvents the narrow-band light filter 220 shown in FIG. 3(a) will be referred to as the “circumvent light path”, and the light path that passes through the narrowband light filter 220 shown in FIG. 3(b) will be referred to as the “filtering light path”. In other words, the movable unit 212 switches the light path of white light emitted by the white LED 206 (or ultraviolet light emitted by the ultraviolet LED 216) between the circumvent light path and the filtering light path.

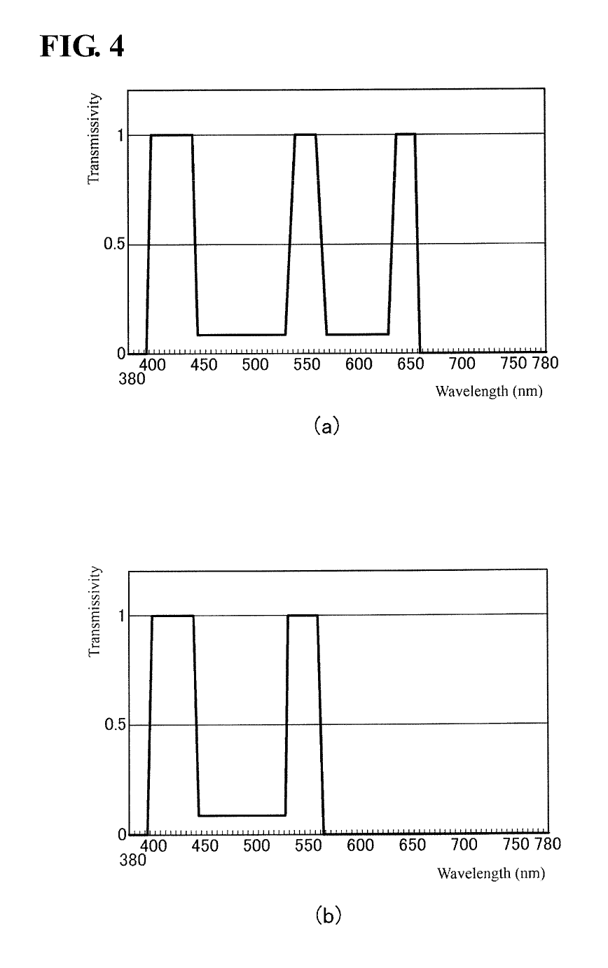

FIG. 4(a) shows an example of the spectral characteristics of the narrow-band light filter 220. Also, FIG. 4(b) shows a different example of spectral characteristics from FIG. 4(a) for the narrow-band light filter 220. As shown in FIGS. 4(a) and 4(b), the narrow-band light filter 220 has a spectral characteristic of allowing only light in a specific wavelength region to pass.

The light that entered the condensing lens 214 is condensed on the entrance surface of an LCB (Light Carrying Bundle) 102 by a condensing lens 214, and enters the LCB 102.

The light that entered the LCB 102 propagates inside the LCB 102. The light that propagated inside the LCB 102 exits from the exit surface of the LCB 102 arranged at the distal end of the electronic endoscope 100, passes through a light distribution lens 104, and irradiates the subject. Returning light from the subject irradiated by the light from the light distribution lens 101 passes through the objective lens 106 and forms an optical image on the light receiving surface of the solid-state image sensor 108.

The solid-state image sensor 108 is a single-plate color CCD (Charge Coupled Device) image sensor that has a Bayer pixel arrangement. The solid-state image sensor 108 accumulates charge according to the light quantity of an optical image formed on pixels on the light receiving surface, generates R (Red), G (Green), and B (Blue) image signals, and outputs the image signals. Note that the solid-state image sensor 108 is not limited to being a CCD image sensor, and may be replaced with a CMOS (Complementary Metal Oxide Semiconductor) image sensor or another type of imaging apparatus. The solid-state image sensor 108 may be an element that includes a complementary color filter.

A driver signal processing circuit 110 is provided in the connection portion of the electronic endoscope 100. Image signals of the subject irradiated by light from the light distribution lens 104 are input by the solid-state image sensor 108 to the driver signal processing circuit 110 at a frame cycle. Note that the terms “frame” and “field” may be switched in the following description. In the present embodiment, the frame cycle and the field cycle are respectively 1/30 seconds and 1/60 seconds. The image signals input from the solid-state image sensor 108 are subjected to predetermined processing by the driver signal processing circuit 110 and output to a pre-stage signal processing circuit 226 of the processor 200.

The driver signal processing circuit 110 also accesses a memory 112 and reads out unique information regarding the electronic endoscope 100. The unique information regarding the electronic endoscope 100 recorded in the memory 112 includes, for example, the pixel count, sensitivity, operable frame rate, and model number of the solid-state image sensor 108. The unique information read out from the memory 112 is output by the driver signal processing circuit 110 to the system controller 202.

The system controller 202 generates control signals by performing various computation based on the unique information regarding the electronic endoscope 100. The system controller 202 uses the generated control signals to control the operations of and the timing of various circuits in the processor 200 so as to perform processing suited to the electronic endoscope that is connected to the processor 200.

A timing controller 204 supplies a clock pulse to the driver signal processing circuit 110 in accordance with timing control performed by the system controller 202. In accordance with the clock pulse supplied from the timing controller 204, the driver signal processing circuit 110 controls the driving of the solid-state image sensor 108 according to a timing synchronized with the frame rate of the images processed by the processor 200.

The pre-stage signal processing circuit 226 performs predetermined signal processing such as demosaicing processing, matrix computation, and Y/C separation on the image signal received in one frame cycle from the driver signal processing circuit 110, and outputs the result to an image memory 228.

The image memory 228 buffers image signals received from the pre-stage signal processing circuit 226, and outputs the image signals to a post-stage signal processing circuit 230 in accordance with timing control performed by the timing controller 204.

The post-stage signal processing circuit 230 performs processing on the image signals received from the image memory 228 to generate screen data for monitor display, and converts the generated monitor display screen data into a predetermined video format signal. The converted video format signal is output to the monitor 300. Accordingly, subject images are displayed on the display screen of the monitor 300.

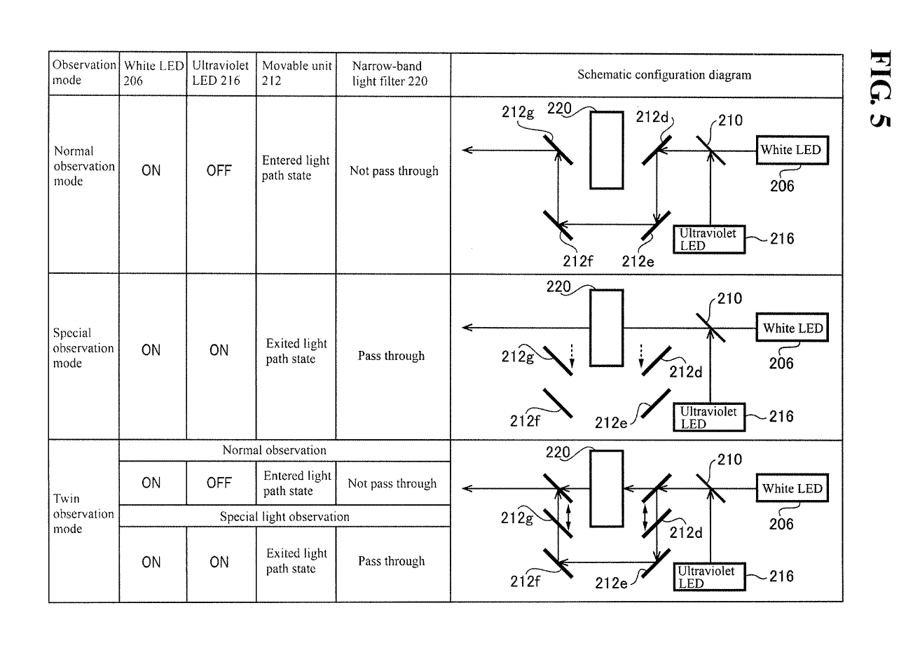

FIG. 5 is a diagram for assisting a description of operations of the electronic endoscope system 1 in various observation modes. Specifically, FIG. 5 shows the ON/OFF states of the LEDs, the operation state of the movable unit 212, the filtering state of the narrow-band light filter 220, and a schematic illustration of various constituent elements (the LEDs, the movable unit 212, and the narrow-band light filter 220) in the various observation modes.

Normal Observation Mode

The following describes operations of the electronic endoscope system 1 in the normal observation mode.

As shown in FIG. 5, in the normal observation mode, the white LED 206 is on at all times, and the ultraviolet LED 216 is off at all times. Also, the movable unit 212 is set to the entered light path state (see FIG. 3(a)). In this case, white light emitted by the white LED 206 travels along the circumvent light path, enters the condensing lens 214, and irradiates the subject. In other words, the subject is irradiated by white light that has the spectral intensity distribution shown in FIG. 2(a).

The solid-state image sensor 108 images the subject irradiated by white light, and outputs the image signal to the pre-stage signal processing circuit 226 via the driver signal processing circuit 110. The image signal is processed by the pre-stage signal processing circuit 226, the image memory 228, and the post-stage signal processing, circuit 230 and then output to the monitor 300, and thus a normal color image of the subject is displayed on the display screen of the monitor 300.

Special Observation Mode

The following describes operations of the electronic endoscope system 1 in the special observation mode.

As shown in FIG. 5, in the special observation mode, the white LED 206 and the ultraviolet LED 216 are on at all times. Also, the movable unit 212 is set to the exited light path state (see FIG. 3(b)). In this case, white light emitted by the white LED 206 and ultraviolet light emitted by the ultraviolet LED 216 travel along the filtering light path, enter the condensing lens 214, and irradiate the subject. In other words, the subject is irradiated by light that is a combination of white light and ultraviolet light (light having the spectral intensity distribution shown in FIG. 2(c)) and has been filtered by the narrow-band light filter 220. Hereinafter, for the sake of convenience in the description, this light that is a combination of white light and ultraviolet light will be referred to as “superimposed light”, and the light filtered by the narrow-band light filter 220 will be referred to as “special light”.

The solid-state image sensor 108 images the subject irradiated by special light, and outputs the image signal to the pre-stage signal processing circuit 226 via the driver signal processing circuit 110. Here, this special light is light that is highly absorbed by a specific biological structure. For this reason, the image signal is processed by the pre-stage signal processing circuit 226, the image memory 228, and the post-stage signal processing circuit 230 and then output to the monitor 300, and thus a spectral image in which a specific biological structure is enhanced is displayed on the display screen of the monitor 300.

Twin Observation Mode

The following describes operations of the electronic endoscope system 1 in the twin observation mode.

In the twin observation mode, the white LED 206 is on at all times. On the other hand, the ultraviolet LED 216 is alternatingly switched on and off (one frame at a time) in accordance with a timing synchronized with the frame cycle. Also, in accordance with a timing synchronized with the frame cycle (one frame at a time), the movable unit 212 is set to the entered light path state when the ultraviolet LED 216 is turned off, and is set to the exited light path state when the ultraviolet LED 216 is turned on. In other words, the light path of irradiation light is alternatingly switched between the circumvent light path and the filtering light path in accordance with a timing synchronized with the frame cycle, which is the imaging cycle, (one frame at a time). In this case, the subject is alternatingly irradiated by white light and special light in accordance with a timing synchronized with the frame cycle (one frame at a time).

In one frame, the solid-state image sensor 108 images the subject irradiated by white light and outputs the image signal to the pre-stage signal processing circuit 226 via the driver signal processing circuit 110, and then in the next frame, images the subject irradiated by special light and outputs the image signal to the pre-stage signal processing circuit 226 via the driver signal processing circuit 110. In other words, the solid-state image sensor 108 alternatingly outputs an image signal of the subject irradiated by white light and an image signal of the subject irradiated by special light to the pre-stage signal processing circuit 226 via the driver signal processing circuit 110. The former and latter image signals are processed by the pre-stage signal processing circuit 226, the image memory 228, and the post-stage signal processing circuit 230 and then output to the monitor 300.

Two regions for displaying observation images are arranged side-by-side in the display screen of the monitor 300. A normal color image of the subject irradiated by white light is displayed in one of the regions, and a spectral image in which the subject irradiated by special light (specific biological structure) is enhanced is displayed in the other region. In other words, a normal color image and a spectral image of the subject are displayed side-by-side on the display screen of the monitor 300.

In this way, according to the present embodiment, the narrow-band light filter 220 is not a moved member, but rather is a member that is fixed inside the case of the processor 200, and therefore there is no need for indicators for detecting the rotation position such as silk lines. Also, because the narrow-band light filter 220 is not a moved member, there are few constraints in terms of its shape, and it may have a simple disk shape for example. In other words, according to the present embodiment, there is no need for indicators required to have strict tolerance management, and there are few constraints on the shape of the narrow-band light filter 220, thereby achieving advantages in terms of manufacturing (e.g., the yield is easily improved).

An illustrative embodiment of the present invention has been described above. The embodiments of the present invention are not limited to the embodiment described above, and various changes can be made without departing from the scope of the technical idea of the present invention. For example, appropriate combinations of embodiments and the like explicitly given as examples in this specification and obvious embodiments and the like are also encompassed in embodiments of the present invention.

The light source apparatus is provided inside the processor 200 in the above embodiment, but in another embodiment, a configuration is possible in which the processor 200 and the light source apparatus are separate. In this case, a wired or wireless communication means for exchanging timing signals is provided between the processor 200 and the light source apparatus.

Also, although the ultraviolet LED 216 is off at all times in the normal observation mode in the above embodiment, the present invention is not limited to this. The ultraviolet LED 216 may be on at all times in the normal observation mode in order to improve color rendering.

Also, although the ultraviolet LED 216 is switched on and off one frame at a time in the twin observation mode in the above embodiment, the present invention is not limited to this. The ultraviolet LED 216 may be on at all times in the twin observation mode in order to improve color rendering.

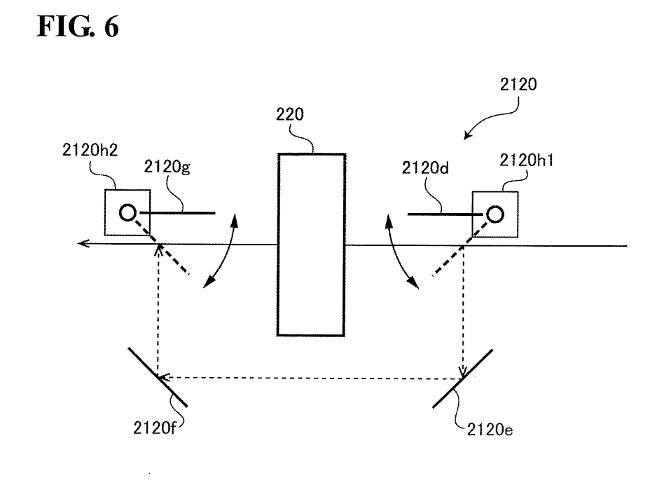

FIG. 6 schematically shows the configuration of a movable unit 2120 according to a variation of the present embodiment. As shown in FIG. 6, the movable unit 2120 includes a first mirror 2120d, a second mirror 2120e, a third mirror 2120f, a fourth mirror 2120g, and actuators 2120h1 and 2120h2.

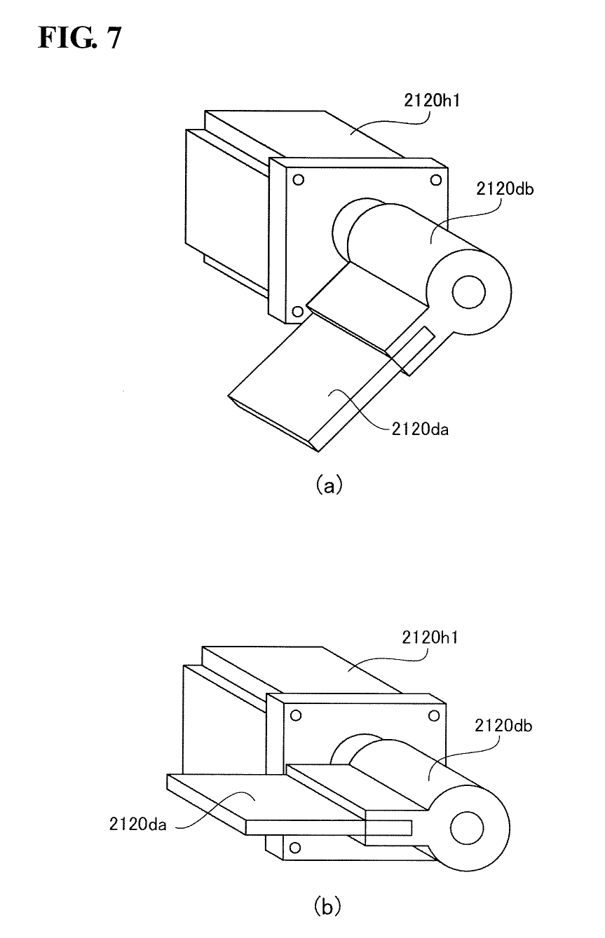

FIG. 7 shows a perspective view of the first mirror 2120d and the actuator 2120h1. As shown in FIG. 7, the first mirror 2120d includes a mirror body 2120da and a mirror holding member 2120db that holds the mirror body 2120da by screw fastening, bonding, or the like. The actuator 2120h1 is a servo motor or a stepping motor, and a drive shaft thereof is press-fitted into a shaft bearing of the mirror holding member 2120db. The first mirror 2120d is rotated about the drive shaft by the actuator 2120h1. Note that the fourth mirror 2120g and the actuator 2120h2 have the same configuration as the first mirror 2120d and the actuator 2120h1, and operate in the same manner.

In the state where the first mirror 2120d and the fourth mirror 2120g have been inserted into the light path (see the first mirror 2120d and the fourth mirror 2120g indicated by dashed lines in FIG. 6. and see FIG. 7(a)), white light (or ultraviolet light) that was incident on the first mirror 2120d1 is reflected by the first mirror 2120d, the second mirror 2120e, the third mirror 2120f, and the fourth mirror 2120g in this order so as to circumvent the narrow-band light filter 220 located between the first mirror 2120d1 and the fourth mirror 2120g, and then enters the condensing lens 214.

On the other hand, in the state where the first mirror 2120d and the fourth mirror 2120g have been removed from the light path (see the first mirror 2120d and the fourth mirror 2120g indicated by solid lines in FIG. 6. and see FIG. 7(b)), white light emitted by the white LED 206 (or ultraviolet light emitted by the ultraviolet LED 216) passes through the narrow-band light filter 220 and then enters the condensing lens 214.

In this way, in the present variation as well, in the former state (see FIG. 7(a) etc.), unfiltered light (light that substantially has the same spectral intensity distribution as when emitted from the LED) enters the condensing lens 214, whereas in the latter state (see FIG. 7(b) etc.), light filtered by the narrow-band light filter 220 enters the condensing lens 214. In the present variation, there is no need for a movable mount or shafts, and the configuration of the moved portions can be suppressed to a small size.

1. A light source apparatus comprising:
a light source;
a switch that switches a light path of irradiation light emitted by the light source between a first light path and a second light path; and
an optical filter that is fixedly arranged in the first light path and filters irradiation light propagating along the first light path into light in a specific wavelength region.

2. The light source apparatus according to claim 1, wherein the switch alternatingly switches the light path of irradiation light between the first light path and the second light path in accordance with a timing synchronized with a predetermined imaging cycle.

3. The light source apparatus according to claim 1,
wherein the switch has a light path changer capable of being inserted into the light path of irradiation light,
the irradiation light enters the second light path when the light path changer is inserted into the light path of irradiation light, and
the irradiation light enters the first light path when the light path changer is removed from the light path of irradiation light.

4. The light source apparatus according to claim 3, wherein the light path changer is a reflecting member that bends the light path of irradiation light.

5. The light source apparatus according to claim 2, wherein the switch inserts the light path changer into the light path of irradiation light or removes the light path changer from the light path of irradiation light by shifting the light path changer in a direction orthogonal to the light path of irradiation light.

6. The light source apparatus according to claim 2, wherein the switch inserts the light path changer into the light path of irradiation light or removes the light path changer from the light path of irradiation light by rotating the light path changer about a predetermined shaft on which the light path changer is supported.

7. The light source apparatus according to claim 1,
wherein the light source apparatus comprises a plurality of the light sources,
a first light source that emits first irradiation light and a second light source that emits second irradiation light are included among the plurality of light sources, and
when the light path of irradiation light is switched between the first light path and the second light path by the switch, the second light source is accordingly switched between on and off states.
